(19) United States
(12) Patent Application Publication
(10) Pub. No.: US 2022/0187292 A1
(43) Pub. Date: Jun. 16, 2022

(54) METHOD AND DEVICE FOR MEASURING THE FIBRINOGEN CONCENTRATION IN BLOOD SAMPLES

(71) Applicants: Jim MANOLIOS, Red Hill (AU); Marek BIALKOWER, Clayton (AU); Clare HENDERSON, Clayton (AU); Rico TABOR, Clayton (AU); Gil GARNIER, Clayton (AU); Heather MCLEISH, Clayton (AU)

(72) Inventors: Jim MANOLIOS, Red Hill (AU); Marek BIALKOWER, Clayton (AU); Clare HENDERSON, Clayton (AU); Rico TABOR, Clayton (AU); Gil GARNIER, Clayton (AU); Heather MCLEISH, Clayton (AU)

(21) Appl. No.: 17/598,080

(22) PCT Filed: Mar. 27, 2020
(86) PCT No.: PCT/AU2020/000024

§ 371 Date: Sep. 24, 2021

(30) Foreign Application Priority Data
Mar. 27, 2019 (AU) 2019901020

(51) Int. Cl. G01N 33/543; G01N 33/86

(52) U.S. Cl. CPC G01N 33/54388; G01N 33/86; G01N 2333/46; G01N 2800/226; G01N 2333/96463; G01N 2333/974; G01N 2800/224; G01N 2333/75

(57) ABSTRACT

A diagnostic device which enables measurement of fibrinogen concentration in a blood sample. The device comprises; a wettable testing substrate including viewing indicators which allow determination of a status of a test. The substrate has a first end and second end and intermediate therebetween a flow receiving zone, a flow path zone and a reaction zone; the reaction zone pre charged with at least one reagent. A blood sample to be tested is deposited near or in either of said flow receiving zone or said reaction zone, the sample reacting with the reagents inducing clotting of the sample. Water added to a dye added to said reaction zone, advances a distance along said substrate. The distance travelled along the substrate by the dye and through the sample is indicative of a measure of concentration of fibrinogen in said blood sample under test.

### BACKGROUND

The present invention relates to diagnostic methodologies and associated equipment and more specifically relates to a diagnostic method which enables measurement of fibrinogen concentration in blood samples. More particularly the present invention also relates to a visual colorimetric method of testing for fibrinogen concentration in a blood sample using a device having an indicator which enables determination of fibrinogen concentration in the sample. The invention further relates to a method of testing of blood samples using fibrinogen and thrombin solutions supplied to a device such as an indicator strip and observing a dyed fluid elution. The invention further relates to method for determination of fibrinogen concentration in a blood sample using a cellulose fiber indicator strip charged with fibrinogen and thrombin solutions and applying a dye droplet to the strip to observe a lengthwise extent of elution.

### PRIOR ART

Fibrinogen is a protein, (a coagulation factor) that is essential for blood clot formation. Fibrinogen is produced by the liver and released into circulation along with several other coagulation factor proteins. Normally, when a body tissue or blood vessel wall is injured, a process called hemostasis begins to arrest bleeding by forming a plug at the injury site. Small cell fragments called platelets adhere to and aggregate at the site and clotting factors are activated one after the other eventually forming a clot.

Fibrinogen is one of those clotting factors. Its concentration can be measured to ascertain a patient's capacity for blood coagulation in the event of a cut or impact injury. As clotting occurs, fibrinogen is converted into insoluble fibrin threads which crosslink together to form a fibrin net that stabilizes at the injury site. The fibrin net adheres to the site of injury along with the platelets to form a stable blood clot. For a stable clot to form there must be enough normally functioning platelets and coagulation factors. If there are dysfunctional factors or platelets, or too little or too much of them, this can lead to bleeding episodes and/or to formation of an in appropriate blood clot—known as thrombosis. It is therefore important to determine clotting capacity in a patient and particularly fibrinogen concentration.

Currently there are a number of tests available to evaluate fibrinogen concentration in the blood and evaluate haemostasis. One such test is a fibrinogen activity test which evaluates how well fibrinogen functions in helping to form a blood clot. A second test known as a fibrinogen antigen test measures the amount of fibrinogen in the blood. The tests are intended to (but do not necessarily) reflect in vivo haemostasis. A laboratory test may not reflect the in vivo behaviour but its aim is to indicate (if not simulate) fibrinogen concentration and it is also useful to evaluate specific components of haemostasis.

The known fibrinogen activity test evaluates that part of the haemostatic process in which soluble fibrinogen is converted into fibrin threads. With the addition of thrombin to the test sample, the fibrinogen test focuses on the function of fibrinogen and measures the time that it takes for a fibrin clot to form following the addition of a standard amount of thrombin to blood plasma. The time that is required for a clot to form directly correlates with the amount of active fibrinogen that is present. This test evaluates the function of fibrinogen, its ability to be converted into fibrin. It is known that prolonged clot-formation times may be due to decreased concentrations of normal fibrinogen or due to dysfunctional fibrinogen.

Another known test methodology is the fibrinogen antigen test which uses a fibrinogen antibody to bind to fibrinogen in a blood sample. This test allows the quantity, but not activity, of fibrinogen to be measured. Concentrations of fibrinogen rise sharply with conditions causing acute tissue inflammation or damage. Tests for these acute phase reactants, including fibrinogen, may be performed using a blood sample to determine the extent of inflammation in the body.

Included in the prior art are the disclosures in US application No. 20120107851A1 which is incorporated by reference herein. That publication teaches a device consisting of a “flow receiving sample” and a “flow path zone” coated with thrombin. Plasma is added to the flow receiving sample and travels through the flow path zone via capillary action where it coagulates and modifies in flow rate. After a set period of time, the plasma stops moving and a distance up the flow path zone provides an indication of fibrinogen concentration. This arrangement works by determining how far the plasma moves through the device while clotting, primarily reliant on micropillar extrusions. The arrangement employs a substrate manufactured from one of plastics, silicon or glass but does not teach any use of cellulose fibre (paper or cardboard).

For example, in the prior art, a plastics diagnostic device is used which has no inherent capillary action to facilitate fluid flow. Accordingly, as plastics diagnostic devices have no capillary action to facilitate fluid flow, it has to be artificially induced by the inclusion of micropillars. Also the plastics diagnostic must be coated with SiOx and treated with polyelectrolytes to increase the hydrophilicity of the flow path zone.

Since this test essentially measures the clot formation time, it is susceptible to changes in thrombin kinetics. The shorter the clot formation time, the higher the fibrinogen concentration. The longer the clot formation time, the lower the fibrinogen concentration. Therefore, plasma samples with levels of thrombin activators are likely to pre-maturely clot the plasma sample and give high fibrinogen concentration readings. Likewise, high concentrations of inhibitors (such as heparin or warfarin) are likely to delay clotting and give falsely low fibrinogen level readings. The latter is especially a problem in western medicine where such inhibitors are used as medications to treat heart attacks.

This disclosure does not teach or allude to an arrangement in which a pigmented solution or dye moves under capillary a distance through clotted plasma after plasma is added to thrombin to react for a period of time and after water is added to the dye or pigment. The prior art does not teach a method in which water wicks a dye through clotted plasma. Nor does it teach a testing device having one part coated with thrombin and another part coated with a dye. Another device for measuring fibrinogen concentration is disclosed in the following publication:

Dudek, M. M., Lindahl, T. L. and Killard, A. J., “Developing a point of care lateral flow device for measuring human plasma fibrinogen”, Analy. Chem., vol 82, 5, pp 2029-2035, March 1 (2010).

This publication teaches a lateral flow assay to measure fibrinogen concentrations using a thermoplastic resin as the substrate. As is described in US20120107851A1, the test described incorporates a flow receiving sample and a flow path zone made of a thermoplastic resin. A flow path zone induces capillary action by the presence of micro pillar. This test pre-immobilises 250-1000 mU of thrombin in the flow path zone and adds 15 μL of plasma preheated at 37° C. to the flow receiving sample. The plasma migrates through the flow path zone where it is clotted. The test stops when plasma movement is arrested. The distance the plasma moves up the flow path zone is correlated to the fibrinogen concentration where higher concentrations lead to lower migrations. The test measures from 0-11 mm for >4 g/L fibrinogen, 12-20 mm for 2-4 g/L fibrinogen and 21-27 mm for 1-2 g/L fibrinogen. The device doesn't reach further than 27 mm length. The mechanism appears to be related to clotting time (as with Clauss Assay). Lower concentration fibrinogen solutions will take longer to form a clot that eventually halts plasma movement. This causes the plasma to travel further up the flow path zone before stopping.

The test method described, which relies of clot formation time, employs micro-structured thermoplastic as a substrate and measures low (1-2 g/L), medium (2-4 g/L) and high (>4 g/L) fibrinogen to cover both haemorrhage and cardiovascular disease risk. Also the prior art method separates 1-2 g/L fibrinogen in mm increments.

There is an ongoing need to constantly seek improvements in and useful alternatives to the known testing regimes to improve testing speed, convenience and efficiency. There is a further need to provide an alternative to and overcome the disadvantages of or at least ameliorate the shortcomings of the known methods to increase the efficiency and speed of Serological testing.

### INVENTION

The present invention provides a diagnostic method which enables measurement of fibrinogen concentration in blood samples using a visual indicator. More particularly the present invention provides a visual colorimetric method of testing for fibrinogen concentration in a blood sample using an indicator which enables determination of fibrinogen concentration in the sample. The invention further provides a method of testing of blood samples using thrombin solutions supplied to an indicator strip and observing a dyed fluid elution. More particularly the invention provides a method for determination of fibrinogen concentration in a blood sample using a cellulose fiber indicator strip charged with thrombin solutions and applying a dye to the strip to observe a lengthwise extent of wicking/elution.

According to an apparatus aspect the present invention provides a handheld diagnostic indicator which may comprise a strip or plate which enables visual measurement of fibrinogen concentration in human blood samples using an increase in hydrophobicity induced by the conversion of fibrinogen to fibrin. According to a method aspect, in a first step, a solution of fibrinogen and thrombin allowing thrombosis to occur, is added to a paper strip. Following that, an aqueous dye is deposited on the strip allowing wicking/elution to occur, thereby enabling a visual indication of fibrinogen concentration of the solution related to a distance the dyed fluid wicks/elutes up each strip.

The visual test enables a determination of concentration of fibrinogen from the length of wicking. The dye is used to improve the visibility of the solution wicking. Preferably the plate or strip is comprised of paper (cellulose fibre). The increase in hydrophobicity is dramatic and depends on the concentration of fibrinogen. If the elution is small the fibrinogen concentration is high and if the elution distance is large (lower hydrophobicity) the concentration is small or very low. According to one embodiment the indicator is a paper strip of a predetermined length and width. Wicking and capillary flow is induced by hydrophilic porous and fibrous media and films.

The parameters which impact on the testing methodolgy and outcomes include: the type of paper or porous media (including wettability and pore size distribution), the strip width, the volume of blood sample added, the individual concentrations of thrombin and FXIIIa used as well as the reaction and elution times. There are 3 other aspects that also have a major effect:
1. The location where the blood sample/thrombin is added on the paper strip (i.e. it is better to add the sample/thrombin further into the flow receiving zone).
2. The width of the flow receiving zone (wider is better).
3. The volume of dye added to the strip (more is better).

Additionally, the effect of non-specific blood proteins on the test to emulate plasma-like conditions is allowed for and quantified. Throughout the description a reference to elution can be taken to include a reference to phenomena including absorption, capillarity. The purpose of an elution is to cause the release of antibody molecules from the red blood cell membrane. Once free in solution the eluted antibodies are tested against reagent red blood cells to determine if an immune antibody specificity is present. Elution is the process of removing antibodies from the surface of red blood cells. Throughout the specification a reference to a plasma sample can be taken to include a reference to blood sample and a reference to blood sample can be taken to include a reference to a plasma sample. Also a reference in the specification to elution can be taken to include a reference to wicking and a reference to wicking can be taken to include a reference to elution. A reference to a kit can be taken to include a reference to a testing device and any associated accessories or simply to a testing device. A reference to clotting can be taken to include a reference to coagulation.

In one broad form the present invention comprises:

a disposable diagnostic device which enables measurement of fibrinogen concentration in a blood sample, the device comprising a wettable porous testing substrate and viewing indicators which allow determination of a test status; the substrate having a first end and second end and intermediate therebetween a flow receiving zone, a flow path zone and a reaction zone; the reaction zone pre charged with Thrombin and an indicating dye or Thrombin and a fibrinogen solution and a dye; wherein when the sample for testing is deposited in said flow receiving zone, or in said reactions zone, porosity in said substrate urges said fluid a distance along said substrate, the distance travelled along said substrate providing a measure of concentration of fibrinogen in said blood sample under test.

According to one embodiment the device includes a housing which includes the viewing windows that indicate to the user along the flow path zone, the fibrinogen concentration

In another broad form the present invention comprises:

a disposable diagnostic strip which enables measurement of fibrinogen concentration in a blood plasma sample applied to said strip, the strip comprising: a wettable porous testing substrate, the substrate having a first end and second end and intermediate therebetween a flow receiving zone, a flow path zone and a reaction zone; the reaction zone pre charged with Thrombin and an indicating dye; wherein when a sample fluid to be tested is deposited in said receiving zone or reaction zone, the sample reacts with said Thrombin inducing clotting; the sample when deposited creating a zone of hydrophobicity; whereby water added to the dye advances along a distance in the flow path zone; the distance travelled along said substrate providing a measure of concentration of fibrinogen in said blood sample under test.

In another broad form the present invention comprises:

a disposable diagnostic indicator device which enables measurement of fibrinogen concentration in a blood plasma sample applied to said device, the indicator comprising: a wettable porous testing substrate, the porous testing substrate having a first end and second end and intermediate therebetween a flow receiving zone, a flow path zone and a reaction zone; the reaction zone of the substrate pre charged with Thrombin and an indicating dye; wherein, when said sample is deposited in said flow receiving zone or in said reaction zone, a zone of hydrophobicity is created; and wherein when water mixes with the dye, the dye advances along the flow path zone; also urging said sample a distance along said substrate, the distance travelled along said substrate of the indicating dye providing a measure of concentration of fibrinogen in said blood sample under test.

In another broad form the present invention comprises:

a device to enable diagnosis of fibrinogen concentration of a blood sample by measuring a hydrophobicity generated in the device after an initiating of clotting of said sample;
the device comprising; a porous testing substrate, the substrate having a first end and second end and intermediate therebetween a flow receiving zone, a flow path zone and a reaction zone; the reaction zone comprised of biological factors, chemical factors and/or derivatives of the biological factors and/or chemical factors pre-applied to the substrate.

In its broadest form the present invention comprises:

a diagnostic device which enables measurement of fibrinogen concentration in a blood sample, the device comprising; a wettable testing substrate including viewing indicators which allow determination of a status of a test; the substrate having a first end and second end and intermediate therebetween a flow receiving zone, a flow path zone and a reaction zone; the reaction zone pre charged with at least one reagent; wherein, when a blood sample to be tested is deposited near or in either said flow receiving zone or said reaction zone, reacts with said reagents inducing clotting of the sample; a dye added to said reaction zone, advances a distance along said substrate; the distance travelled along said substrate by the dye and through the sample providing a measure of concentration of fibrinogen in said blood sample under test.

According to a preferred embodiment the porous substrate is manufactured from cellulose fibre (paper). According to a preferred embodiment, the reaction zone is pre charged with Thrombin and an indicating dye; wherein when a sample fluid is deposited in said flow receiving zone, porosity in said substrate urges said fluid under the action of capillary, a distance along said substrate. A dye mixed with water advances along the substrate in the flow path zone with the distance of travel related of the dye providing an indicator from which concentration of fibrinogen in said blood sample under test can be ascertained.

According to one embodiment, the wettable porous substrate is modified with a physical factor and/or chemical factor or biological factor that will or may increase or decrease the hydrophobicity of the substrate. In one embodiment the chemical factor modification of the substrate comprises coating the substrate. The blood plasma is according to one embodiment applied on the porous substrate outside the reaction zone. Alternatively the blood or plasma is applied to the reaction zone to form a zone of hydrophobicity after initiating clot formation. Measuring the hydrophobicity refers to measuring the zone of hydrophobicity's hydrophobicity.

The aforesaid biological factors, chemical factors and/or derivatives of the biological factors and/or chemical factors used are involved in the initiation, execution, amplification and/or acceleration of the clot formation. Alternatively, the biological factors, chemical factors and/or derivatives of the biological factors and/or chemical factors used are involved in the enhancement or diminishment of the clot's hydrophobicity. The biological factors, chemical factors and/or derivatives of the biological factors and/or chemical factors used may be applied and/or pre-applied outside of the reaction zone. The aforesaid physical factors used will or may allow or prevent the initiation of the clot formation. Physical factors used may enhance or diminish the clot's hydrophobicity. Alternatively any physical factors used that will or may increase or decrease the execution, amplification and/or acceleration of the clot formation.

In another broad form the present invention comprises:

a method of diagnosis of concentration of fibrinogen in a blood or plasma sample, using at least one capillary in a centrifuge, said at least one capillary each including a reaction mixture of biological factors, chemical factors and/or derivatives of the biological factors and/or chemical factors applied and/or pre-applied inside of the at least one capillary, the method including the step of applying blood or plasma to said reaction mixture to form a clot and measuring an extent of clotting after initiating clotting of the sample. Measuring the extent of clotting refers to quantifying the mass, volume or height of the clot in the capillary. An indicating dye is mixed with water and advances along a flow path with the distance advanced determinant of concentration of fibrinogen in the bold sample.

In another broad form according to a method aspect, the present invention comprises: a testing method for determining the concentration of fibrinogen in a test sample using a porous substrate; the substrate having a first end and second end and intermediate therebetween a flow receiving zone, a flow path zone and a reaction zone; the method comprising the steps of:
a) pre charging said porous substrate with Thrombin chromogenic substance and an indicating dye, in the reaction zone;
b) adding a plasma/blood sample to said reaction zone;
c) allowing the plasma/blood to react with the thrombin and undergo clotting;
d) allowing said plasma to create a zone of hydrophobicity,
e) adding water to the dye and allowing the dye to advance along the substrate to a distance;
f) measuring said distance to determine fibrinogen concentration of said plasma sample.

Preferably the method of quantifying the zone of hydrophobicity/hydrophobicity consists of measuring the distance travelled by at least one chromogenic marker through or away from the zone of hydrophobicity in a lateral flow set-up.

In other broad form of the method aspect, the present invention comprises: a method of testing for the concentration of fibrinogen in a blood sample using a diagnostic device which enables measurement of fibrinogen concentration in a blood sample, the device comprising; a wettable testing substrate and a housing including viewing indicators which allow determination of a status of a test; the substrate having a first end and second end and intermediate therebetween a flow receiving zone, a flow path zone and a reaction zone; the reaction zone pre charged with reagents;

the method comprising the steps of:
a) pre charging said porous substrate with Thrombin chromogenic substrate and a dye/buffer solution, to provide a reaction mixture in a reaction zone;
b) adding a blood or plasma sample near or in said receiving zone or near or in said reaction zone so that is engages with;
c) allowing the plasma to react with the thrombin in the reaction mixture to create a zone of hydrophobicity;
d) using the porosity in the substrate to transport the dye/buffer solution along the substrate and through the reaction zone,
e) observing a distance along the porous substrate that the dye/buffer solution passes;
f) determining fibrinogen concentration in said sample with reference to said distance that the dye/buffer solution travels along the flow path zone
g) allowing blood or plasma sample to form a clot and measuring an extent of clotting after initiating clotting of the sample, by measuring mass or volume or height of the clot.

In another broad form of the method aspect, the present invention comprises:

a testing method for determining the concentration of fibrinogen in a test sample using a porous substrate; the substrate having a first end and second end and intermediate therebetween a flow receiving zone, a flow path zone and a reaction zone; the method comprising the steps of:
h) pre charging said porous substrate with Thrombin chromogenic substrate to provide a reaction zone and an indicating dye;
i) adding a plasma sample to said reaction zone;
j) allowing the plasma to react with the thrombin to clot and create a zone of hydrophobicity;
k) washing the dye/buffer solution in the reaction zone,
l) observing a distance that the dye/buffer solution travels along the flow path zone and colour change;
m) determining fibrinogen concentration in said sample with reference to said distance and/or said colour change.

The hydrophobicity is induced by the polymerization of the fibrinogen in the blood sample to fibrin upon enzymatic reaction with thrombin and/or FXIIIa deposited onto the receiving surface. According to one embodiment diagnosis relies on a significant change of hydrophobicity induced by polymerization of the fibrinogen in the blood sample to fibrin upon enzymatic reaction with thrombin and/or FXIIIA deposited in the receiving zone. The method of quantifying the zone of hydrophobicity's surface hydrophobicity consists of measuring the shape, height and/or contact angle of any deposited liquid droplets on top of the non-porous substrate's surface. According to one embodiment, capillarity distributes the blood sample into the receiving zone treated with thrombin, and a wash solution is transported through the receiving zone to remove dye deposited in the receiving zone out of the porous material. The colour intensity after washing is used to measure and visualize the hydrophobicity of the zone. Therefore, colour intensity is correlative to the fibrinogen concentration in the blood sample.

According to an alternative embodiment of the method aspect, a diagnosis of fibrinogen concentration can be made using the following combined three mechanisms. The first mechanism is a change in dye adhesiveness induced by polymerization of the fibrinogen in the blood sample to fibrin upon enzymatic reaction with thrombin and/or FXIIIa deposited onto the receiving zone. The second mechanism is the adhesion of the dye deposited in the receiving zone to the fibrin directly (or indirectly via the assistance of dye binders). The third mechanism is capillarity used to: 1) distribute the blood sample into the receiving zone treated with thrombin, and 2) transport wash solution through the receiving surface to remove dye out of the porous material. The colour intensity after washing is used to measure and visualize the quantity of fibrin-adhered dye remaining in the zone. Therefore, colour intensity is correlative to the fibrinogen concentration in the blood sample.

According to a preferred embodiment, said indicator or said strip comprises cellulose fibre or other suitable porous and wettable material. Preferably a blood sample is deposited in the reaction zone of the indicator device and is allowed to react with a precharge of Thrombin. The indicator is preferably pre-charged with Thrombin and/or FXIIIa or similar. The indicator is either pre charged with a dye/buffer solution to the reaction zone or to a different receiving zone. Alternatively, the dye is added during or after introduction of the blood/plasma sample. The indicative dye or solution may contain a buffer. The dye allows a visual indication of distance that fluid added to the flow receiving zone travels dictated by the zone of hydrophobicity which is in turn dictated by the reaction between a plasma sample and Thrombin. This allows a diagnostic result for Fibrinogen concentration based on the measured distance.

Preferably said dye in said reaction zone, acts as a visual indicator and is blue in colour. Preferably said Thrombin is Lyophillised. Preferably said reaction zone is in said flow path zone. Plasma advances along the substrate through capillary action.

Results suggest that the reaction zone (and the eventual zone of hydrophobicity) is better spread across both the flow path and flow receiving zone.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying illustrations and descriptive matter in which there is illustrated various including preferred embodiments of the invention.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION OF DRAWINGS

The invention will be better understood and objects other than those set forth above will become apparent when consideration is given to the following detailed description thereof. Such description will now be described in more detail according to a preferred but non-limiting embodiments and with reference to the accompanying illustrations; wherein

FIG. 1 shows a schematic arrangement of a fibrinogen concentration diagnostic testing device including a porous substrate.

FIG. 2 shows the device of FIG. 1 in a first stage of testing

FIG. 3 shows the device of FIG. 1 at a second stage of testing.

FIG. 4 shows the testing device of FIG. 1 with an outer casing completing a testing kit and with visual indicators indicating an unused state.

FIG. 5 shows the casing of FIG. 4 indicating a first stage of testing with visual indicators indicating a reaction and a valid test.

FIG. 6 shows the device of FIG. 4 at a second stage of testing with visual indicators indicating a result.

### DETAILED DESCRIPTION

The present invention will now be described in more detail according to a preferred embodiment but non limiting embodiment and with reference to the accompanying illustrations. The examples referred to herein are illustrative and are not to be regarded as limiting the scope of the invention. While various embodiments of the invention are described herein, it will be appreciated that these are capable of modification, and therefore the disclosures herein are not to be construed as limiting of the precise details set forth, but to avail such changes and alterations as fall within the purview of the description.

### Terminology

Throughout the description a reference to substrate can be taken to include a reference to: Active Base/medium of diagnostic which facilitates assaying. A reference to Factor can be taken to include a reference to: a component of diagnostic that impacts on results. a reference to Biological factor can be taken to include a reference to: a Factor derived from biological source. Eg. Thrombin, Platelets, etc. . . . . A reference to Chemical Factor: can be taken to include a reference to: a factor derived from non-biological source. Eg. Calcium Chloride, etc. . . . . A reference to physical factor can be taken to include a reference to: a Factor: not composed of chemical matter. Eg. UV Radiation, Temperature, Pressure.

A reference to apply/applied can be taken to include a reference to: the addition of a chemical to diagnostic as a part of test procedure. A reference to pre-apply can be taken to include a reference to: incorporation of a chemical into diagnostic as part of intrinsic design. A reference to clot can be taken to include a reference to: a Network composed of polymerized fibrin monomers and a reference to clotting or clot formation can be taken to include a reference to Conversion of fibrinogen into fibrin network. A reference to Amplification (of clot formation) can be taken to include a reference to: generation of a larger magnitude clot network in the time allowed for reacting.

A reference to Chromogenic Marker, also termed a dye, can be taken to include a reference to: a coloured chemical that is clearly visible to the naked eye. A reference to Lateral Flow can be taken to include a reference to: movement of liquid through a long, narrow channel via capillary forces. A reference to Wicking Liquid can also be taken to include a reference to: a liquid that moves through a substrate through the action of capillary forces. A wicking fluid is also a fluid that saturates the flow receiving zone in order to travel through the flow path zone.

A reference to a flow receiving zone can be taken to include a reference to: a zone in which wicking liquid can be applied. A reference to a flow path zone, can be taken to include a reference to: a Zone in which wicking liquid can move into from the flow receiving zone.

A reference to a Washing Liquid, can be taken to include a reference to: a liquid used to dislocate the chromogenic marker(s) out of the substrate. The plasma/blood solution too can be defined as washing liquid in this case as it is able to dislocate the dye out of the paper (if the dye is pre-charged Into the reaction zone).

A reference to a, Threshold Result can be taken to include a reference to: a simplified quantification in which the result provided from a measurement is only readable as above or below a certain value (i.e. the reading is positive or negative).

A reference to a, surface (of substrate) can be taken to include a reference to: a solid interface of substrate's surface that is only exposable to liquid or gas; includes any chemical modifications or coatings applied and/or pre-applied to this interface. A reference to Capillary can be taken to include a reference to: a thin tube which can hold blood or plasma. A reference to a centrifuge or centrifuge device, can be taken to include a reference to: a device that applies centripetal or centrifugal forces (to a sample).

The invention is described herein with reference to paper strips for the purpose of illustration but it will be appreciated by persons skilled in the art that the invention has applications apart from fibrinogen testing. Throughout the description a reference to wicking can be taken to include a reference to acting to absorb or draw off liquid by capillary.

According to one embodiment there is provided a paper indicator using thrombin and a dyed solution to ascertain concentration of fibrinogen in a blood sample using distance increments which can potentially be measured in mm or cms. The indicator is intended for measuring between 0-2 g/L to diagnose hypofibrinogenemia (especially in the early stages of hemorrhage). According to the method aspect, the test employs plasma and measures based on clot hydrophobicity rather than clot formation time as in the known art.

According to a preferred embodiment a paper substrate is provided which has one section coated with thrombin and another section coated with blue dye. Plasma is added to the section with thrombin to react for a short period of time, then water is added to the section with blue dye. It is preferred to use the blue dye in the reaction zone (and causing it to wick in the eventual zone of hydrophobicity) instead. The distance that is measured is how far that the water wicks the blue dye through the clotted plasma. In reality it also moves the zone of hydrophobicity with it (whilst simultaneously moving through it). This is to be distinguished from the prior art which measures how far the plasma moves through the device whilst clotting. The front of the zone of hydrophobicity can be used as a distance marker as it can be much clearer than that produced by the wicking fluid moving through it.

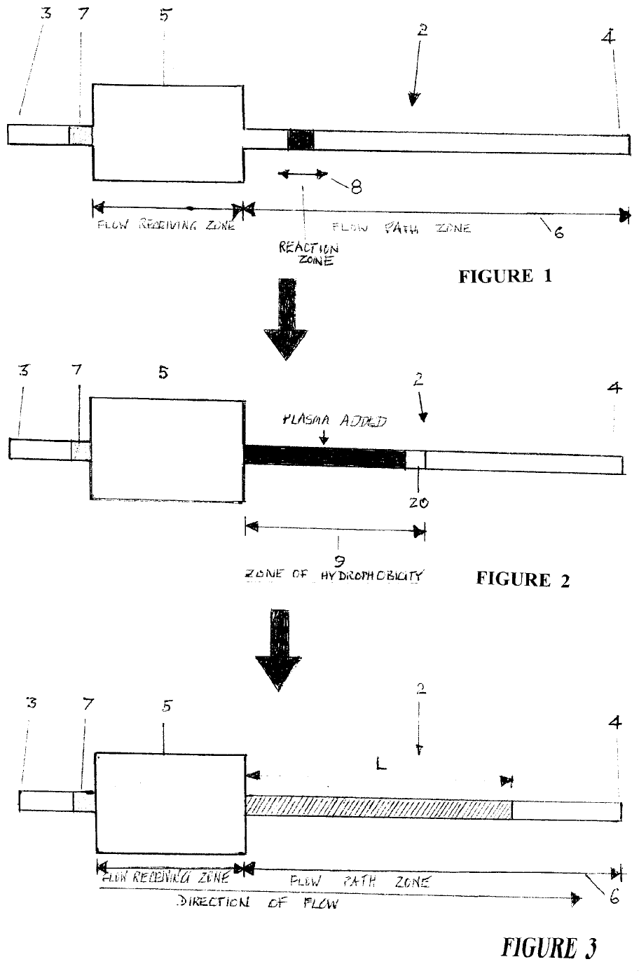

FIG. 1 shows according to one embodiment, a schematic arrangement of a fibrinogen concentration diagnostic testing device 1 (see FIG. 2) including a porous substrate 2. Preferably substrate 2 is provided in the form of a strip but it will be appreciated that other geometries are feasible as long as it includes a flow path zone. According to the embodiment shown, substrate 2 comprises a first end 3 and second end 4. In between ends 3 and 4 are a flow receiving zone 5 and a flow path zone 6 for analyzing test results. Substrate 2 includes in the flow path zone 6 a Thrombin Chromogenic substrate (for example S2238). [Chromogenic substrates are peptides that react with proteolytic enzymes under the formation of color. They are made synthetically and are designed to possess a selectivity similar to that of the natural substrate for the enzyme. A chromogenic substrate is acted on by an enzyme so as to increase or decrease the absorption of light at a particular wavelength as the substrate is converted to product.] Adjacent flow receiving zone 5 for receiving water and near end 3 is a wax boundary 7 which provides a flow limit for water introduced into the flow receiving zone 5 during testing. Substrate 2 further comprises a reaction zone 8 which preferably incorporates Lyophilised thrombin and a blue dye. Reaction zone 8 is pre charged with Thrombin and the dye.

FIG. 2 shows with corresponding numbering the substrate device 2 of FIG. 1 in a first stage of testing to determine fibrinogen concentration in a blood plasma sample. According to one embodiment, plasma is added to reaction zone 8 preferably using a pipette. Capillary forces in the substrate 2 wicks the plasma, blue dye and thrombin laterally in the direction of either of ends 3 and/or 4. Thrombin reacts with plasma fibrinogen and the thrombin chromogenic substrate. This creates a zone of hydrophobicity 9. Indication zone 20 undergoes a colour change as Thrombin cleaves the Chromogenic substrate.

FIG. 3 shows the device of FIG. 1 at a second stage of testing. During this stage, water is added to flow receiving zone 5. This may be done using a pipette or alternatively placing the substrate 2 into a beaker of a wicking fluid such as but not limited to water. Capillary forces in porous substrate 2 urges/wicks the wicking fluid along flow path zone 6. A distance L that the wicking fluid travels along flow path 6 is a function of a level of hydrophobicity in the zone of hydrophobicity 9. The distance that the water travels along the substrate is a visual indication of the fibrinogen concentration in the plasma sample.

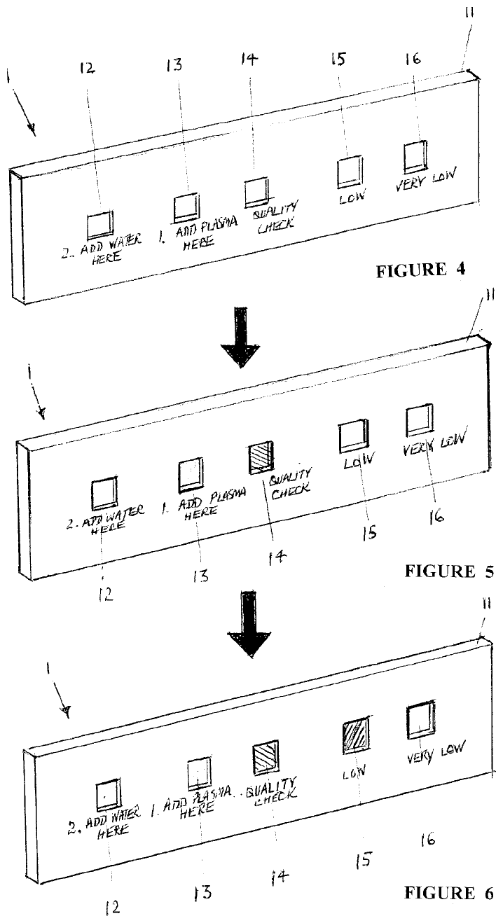

FIG. 4 shows an unused testing device 1 comprising substrate 2 of FIG. 1 inside and housed in an outer/housing/casing 11 completing testing device 1 and with visual indicators indicating an unused state. Casing 11 is divided into application indicator windows and analysis windows. In the application windows are provided an indication window 13 which indicates plasma introduction and window 12 which indicates location of water introduction. Upon addition of plasma, window 13 undergoes a colour change. A quality check window 14 is provided which is used to assess pre reaction of a plasma/blood sample and Thrombin. Window 14 posses the thrombin Chromogenic substrate. When thrombin is spread to it after the addition of the plasma or blood sample (from window 13), it changes colour to indicate that the thrombin hasn't degraded indicating that the test is valid. Zone 6 (which contains the uncleaved thrombin chromogenic substrate) lies under Window 14. When the blood or plasma sample is added to window 13, it allows the pre-charged thrombin from the reaction zone to spread to zone 6. The inactive thrombin chromogenic marker can be placed in a separate area upstream from the reaction zone. This can be denoted by an extra box in FIG. 4 (not shown) without shading to indicate its lack of colour instead. Then when the plasma/blood sample is added, it becomes coloured as indicated in FIG. 5. If the thrombin is working, it will then cleave the thrombin chromogenic substrate, and cause window 14 to change colour to indicate a valid test. If the thrombin isn't working, then no colour change will occur and it will indicate to the user that the test device cannot be used. At completion of testing, window 15 will provide an indication of a low fibrinogen or window 16 will provide an indication of a very low fibrinogen. This is preferably effected by a colour indication appearing in either window 15 or 16.

FIG. 5 shows with corresponding numbering, the housing/casing 11 of FIG. 4 indicating a first stage of testing with visual indicator quality check window 14 indicating a reaction has occurred between the sample and Thrombin chromogenic substrate and that there is a valid test (quality check). The indication of a valid test in window 14 is preferably achieved by a colour change—for example, but not limited to, white to green. A dye can also be incorporated into the reaction zone. Zone 6 (which contains the uncleaved thrombin chromogenic substrate) lies under Window 14. When the blood or plasma sample is added to window 13, it allows the pre-charged thrombin from the reaction zone to spread to zone 6. If the thrombin is working, then it will cleave the thrombin chromogenic substrate, and cause window 14 to change colour to indicate a valid test. If the thrombin isn't working, then no colour change will occur and it will indicate to the user that the test cannot be used. When the blood/plasma sample is added, it will spread the dye under window 14 as well. Hence the window 14 after plasma addition may appear blue initially before turning green once the thrombin chromogenic substrate has reacted.

FIG. 6 shows the indications at a second stage of testing. Referring to indicator windows, window 13 indicates that plasma was added and that from window 14 a valid test was performed. In this case window 16 shows no indication (remains white) but window 15 shows that the patient tested has a low concentration of Fibrinogen between 1-2 g/L. of device of FIG. 4 at a second stage of testing with visual indicators indicating a result. In this case Fibrinogen concentrate is needed.

The present invention employs paper, cellulose or any porous and wettable material as a medium for both the plasma and water to wick through. The porous structure of the cellulose induces a capillary action but without reliance on action from micropillar extrusions. Paper is economic in comparison to plastics, glass or silicon (AU$5 cents per test vs AU$50 cents per test). Paper is a flexible material that can be cut into many different shapes, configurations and structures and can be easily incorporated with hydrophobic barriers and hydrophilic channels. Therefore, its fabrication costs are very economic compared to the prior art.

The present invention presents many advantages over the prior art. For example, in the prior art, a plastics diagnostic device is used which has no inherent capillary action to facilitate fluid flow. Accordingly, fluid flow must be artificially induced by the inclusion of micropillars. In addition, the prior art plastics diagnostic device needs to be coated with SiOx and treated with polyelectrolytes to increase the hydrophilicity of the flow path zone as in US20120107851A1. The extra manufacturing adds significantly to the costs of producing the device. According to the present invention, a paper indicator is used which requires no pre treatment to induce capillarity as the material itself has inherent capillarity requiring no pretreatment or modification to induce capillarity. One advantage of the present invention is that use of a paper indicator, has natural capillary flow without the need to add in parts or modify surfaces.

Another advantage is that the disadvantages of variability related to thrombin kinetics is eliminated. Furthermore, the employment of a porous material such as paper, allows fibrinogen concentration to be measured in other ways than a lateral flow assay. For example, the test can be converted into a flow-through type detection system that measures colour intensity after a certain number of washes. Another advantage of the present invention is that the sensitivity for the test is higher for concentration ranges of 1-2 g/L Fibrinogen. For example in the cited prior art US20120107851A1 a distance travelled for between 1 and 2 g/L plasma is only 0.6 cm before stopping.

According to the present invention there is for example, a 1.7 cm separation for the same concentrations after 7 minutes of elution. It is contemplated that substrate 2 includes a variety of user options for wicking fluid, including a wetable fibrous material and includes but is not limited to, non wetable materials treated by plasma treatment, radiation, surfactant coating and/or chemical reaction to make it wettable. The substrate may be woven or non-woven. It may be pre or post treated with thrombin and/or FXIIIa or derivatives of these enzymes. It may be treated with deposited desorbable dyes and/or desorbable dye binders and particles and nanoparticles. During testing the substrate is charged with water, buffer solution, dye solution and/or washing solution—collectively wicking fluids.

There are provided alternative methods for analyzing the testing and test results. A method of quantifying the zone of hydrophobicity/hydrophobicity includes measuring the distance travelled by at least one chromogenic marker with the zone of hydrophobicity 9 in a lateral flow. Preferably the lateral flow occurs in porous substrate 2 in flow receiving zone 5 and flow path zone 6. According to one embodiment, flow path zone 6 has a length L1 and a width W1. Flow receiving zone 5 has a length L2 and a width W2. L1 may be the same or different length from L2. Likewise W1 may be the same or a different length from W2. A lateral flow regime traverses the flow receiving zone 5 and flow path zone 6 and as described earlier. According to one embodiment, the biological factors, chemical factors and/or derivatives of the biological factors and/or chemical factors pre charge substrate 2 in flow path zone 6. Alternatively those factors may be applied during testing to at least part of the flow path zone 6 and/or the flow receiving zone 5. In use, a blood or plasma sample is introduced into either at least part of the flow path zone 6 and/or the flow receiving zone 5. Chromogenic marker(s) can be applied at this time or pre-applied to the substrate 2.

A wicking fluid is applied to the flow receiving zone 5 in order to induce the movement of the chromogenic marker(s) through the flow path zone 6 The wicking fluid is according to one embodiment applied to the flow receiving zone in the form of a finite reservoir or an infinite volume reservoir. The distance travelled by the chromogenic marker(s) is measured with ruling markers next to the flow path zone 6. When substrate 2 is held in casing 11 to form device 1, the distance travelled by the chromogenic marker(s) is/are measured via by observing the flow path zone 6 through transparent windows 12, 13, 14, 15 and 16 in casing 11. Windows 12, 14, 15 and 16 align with the flow path zone 6 and enable the observations described earlier with reference to FIGS. 5 and 6. The indicating windows align with key distances up the flow path zone 6 and will show wherever the chromogenic marker(s) have reached and/or gone past the key distances or not.

According to one embodiment, hydrophobicity of the zone of hydrophobicity 9 is determined by measurement of degree of retention of at least one chromogenic marker applied and/or pre-applied in (or outside of) the reaction zone, along with all the other biological factors, chemical factors and/or derivatives of the biological factors and/or chemical factors, after rinsing the porous substrate with a washing liquid. Hydrophobicity refers to the hydrophobicity at the surface of the non-porous substrate. The substrate's surface hydrophobicity is used to influence shape, height and/or contact angle of any deposited liquid droplets. Hydrophobicity can be modified with a physical factor and/or chemical factor that will or may increase or decrease the hydrophobicity of the substrate. One method of modifying the hydrophobicity is to apply a chemical coating to the substrate.

Preferably the clot formed acts as a hydrophobic barrier to prevent the washing liquid from dislocating of the chromogenic marker(s) out of the substrate. The forming or formed clot can coat the surface of the non-porous substrate to modify its hydrophobicity. Physical factors may allow or prevent the initiation of the clot formation. Physical, Biological factors, chemical factors and/or derivatives of the biological factors and/or chemical factors used are involved in the initiation, execution, amplification and/or acceleration of the clot formation. These factors also affect the clot's hydrophobicity. The retention of chromogenic marker(s) is measured by the absolute and/or relative colour intensity after washing the substrate with a given volume of washing liquid. The chromogenic marker(s) is applied and/or pre-applied in the reaction zone, along with all the other biological factors, chemical factors and/or derivatives of the biological factors and/or chemical factors.

There are many variations in the methodology of performing the fibrinogen concentration testing. The variations include:

applying blood or plasma on the non-porous substrate outside the reaction zone; application of the blood or plasma to the reaction zone to form a zone of hydrophobicity after initiating clot formation;
removing clotted blood or plasma from the non-porous substrate's surface by physical factors;
enhancing the visibility of the liquid droplet with the use of at least one chromogenic maker . . .
a threshold result for height measurements which can be determined by placing a porous, absorbent substrate directly above the non-porous substrate and allowing any deposited liquid droplet to make contact with the porous, absorbent substrate.

The method contemplates a process of measuring chromogenic staining which refers to the binding of at least one chromogenic substrate directly and/or indirectly to the formed or forming clot. Blood or plasma is applied to the reaction zone to form a zone of stained clot after initiating clot formation. Biological factors, chemical factors and/or derivatives of the biological factors and/or chemical factors can be used enhance the colour intensity of the chromogenic marker(s).

According to an alternative embodiment, the present invention one method of diagnosis of concentration of fibrinogen in a blood or plasma sample, involves using at least one capillary in a centrifuge, each including a reaction mixture of biological factors, chemical factors and/or derivatives of the biological factors and/or chemical factors applied and/or pre-applied inside of the at least one capillary. Blood or plasma is applied to the reaction mixture which forms a clot and the extent of clotting after initiating clotting is measured. Measuring the extent of clotting refers to quantifying the mass, volume or height of the clot in the capillary.

A centrifugal force is applied to the capillaries such that it causes the clot to compress in the direction away from the axis of spin. The extent of clotting is determined by measuring the height of the centrifugally compressed clot in the capillary. The height of the compressed clot can be measured with ruling markers on top of and/or next to the capillar(ies). Alternatively the compressed clot is measured by containing the capillary in an external casing with transparent windows that align with key heights up the capillary and hence will show whether the clot has reached and/or gone past the key heights or not.

As previously described, a chromogenic dye or other suitable marker is used. Biological factors, chemical factors and/or derivatives of the biological factors and/or chemical factors are used enhance the colour intensity of the chromogenic dye(s) and are involved in the initiation, execution, amplification and/or acceleration of the clot formation. Physical factors used that will or may allow or prevent the initiation of the clot formation. the biological factors, chemical factors and/or derivatives of the biological factors and/or chemical factors used are involved in the enhancement or diminishment of the clot's hydrophobicity. Physical factors used that will or may enhance or diminish the clot's hydrophobicity. The biological factors, chemical factors and/or derivatives of the biological factors and/or chemical factors used, are involved in the enhancement or diminishment of the clot's ability to bind directly and/or indirectly to at least one chromogenic marker.

It will be recognised by persons skilled in the art that numerous variations and modification may be made to the invention broadly described herein without departing from the overall spirit and scope of the invention.

The claims defining the invention are as follows:

1. A diagnostic device which enables measurement of fibrinogen concentration in a blood sample, the device comprising; a wettable testing substrate including viewing indicators which allow determination of a status of a test; the substrate having a first end and second end and intermediate therebetween a flow receiving zone, a flow path zone and a reaction zone; the reaction zone pre charged with at least one reagent; wherein, when a blood sample to be tested is deposited near or in either of said flow receiving zone or said reaction zone, the sample reacting with said reagents inducing clotting of the sample; water added to a dye added to said reaction zone, advances a distance along said substrate; the distance travelled along said substrate by the dye and through the sample providing a measure of concentration of fibrinogen in said blood sample under test.

2. A device according to claim 1 wherein a zone of hydrophobicity is created in or near said reaction zone when blood or plasma is applied to the reaction zone.

3. A device according to claim 2 wherein the reaction zone pre charged reagents are selected from the group comprising; physical factors, biological factors, and chemical factors pre applied to the substrate.

4. A device according to claim 5 wherein, the concentration of fibrinogen is related to the behavior of the blood sample introduced into the fluid receiving zone or reaction zone and a hydrophobicity induced into the substrate.

5. A device according to claim 4 wherein the substrate is porous and manufactured from cellulose fibre (paper).

6. A device according to claim 5 wherein when the sample is deposited in or near said flow receiving zone or reagent zone; the porosity of said substrate urging said dye under the action of capillary (elution), a distance along said substrate, the distance travelled along said substrate and through said sample, providing a measure of concentration of fibrinogen in said blood sample under test.

7. A device according to claim 6 wherein said elution causes a release of antibody molecules from the red blood cell membrane of the test sample.

8. A device according to claim 7 wherein said released antibodies are tested against reagent red blood cells to determine if an immune antibody specificity is present.

9. A device according to claim 8 wherein the physical factor and/or chemical factor or biological factor applied to the porous substrate, influences hydrophobicity of the substrate.

10. A device according to claim 9 wherein the zone of hydrophobicity is measurable.

11. A device according to claim 10 wherein the physical factor and/or chemical factor or biological factor applied to the porous substrate, decreases or increases hydrophobicity of the substrate.

12. A device according to claim 11 wherein the pre charging of chemical factor modification of the substrate comprises coating the substrate with a chemical.

13. A device according to claim 12 wherein when blood or plasma is applied to or near the reaction zone, a zone of hydrophobicity is formed after initiation a clot formation.

14. A device according to claim 13 wherein the biological factors, physical factors, chemical factors and/or derivatives of the biological factors and/or chemical factors used may optionally be applied and/or pre-applied outside of the reaction zone.

15. A device according to claim 14 wherein the blood plasma is applied on the porous substrate outside the reaction zone.

16. A device according to claim 15 wherein, the aforesaid biological factors, physical factors, chemical factors and/or derivatives of the biological factors, physical factors and/or chemical factors used are involved in the initiation, execution, amplification and/or acceleration of the clot formation.

17. A device according to claim 16 wherein the aforesaid biological factors, physical factors, chemical factors and/or derivatives of the biological factors, physical factors and/or chemical factors used are involved in the enhancement or diminishment of the hydrophobicity of the clot.

18. A device according to claim 17 wherein the physical factors used influence hydrophobicity of the clot.

19. A device according to claim 18 wherein the aforesaid physical factors determine the creation or prevention of clot formation.

20. A device according to claim 19 wherein the device is single use and disposable.

21. A device according to claim 20 wherein the reaction zone is pre charged with Thrombin chromogenic substrate and a dye.

22. A device according to claim 21 wherein the hydrophobicity is induced by the polymerization of fibrinogen in the blood sample to fibrin upon enzymatic reaction with thrombin and/or FXIIIa deposited onto the receiving or reaction zones.

23. A device according to claim 22 wherein diagnosis relies on a significant change of hydrophobicity induced by polymerization of the fibrinogen in the blood sample to fibrin upon enzymatic reaction with thrombin and/or FXIIIA deposited in the receiving or reaction zone.

24. A device according to claim 23 wherein capillarity distributes the blood sample into the receiving zone treated with thrombin, and a wash solution is transported through the receiving zone to remove dye deposited in the receiving zone out of the porous material.

25. A device according to claim 24 wherein colour intensity after washing is used to measure and visualize the hydrophobicity of the zone and relate to the fibrinogen concentration in the blood sample.

26. A device according to claim 25 wherein the porous substrate is contained in a housing including viewing indicators.

27. A device according to claim 26 wherein the viewing indicators are observed through viewing windows that reveal status of testing as the indicator dye and/or sample progresses along the substrate.

28. A device according to claim 27 wherein the distance is a measurement of how far that the water wicks blue dye through clotted plasma.

29. A device according to claim wherein the blue dye moves the zone of hydrophobicity with it (whilst simultaneously moving through it).

30. A disposable diagnostic indicator device which enables measurement of fibrinogen concentration in a blood plasma sample applied to said device, the indicator comprising: a wettable porous testing substrate, the porous testing substrate having a first end and second end and intermediate therebetween a flow receiving zone, a flow path zone and a reaction zone; the reaction zone of the substrate pre charged with Thrombin and an indicating dye; wherein, when said sample is deposited in said flow receiving zone or in said reaction zone, a zone of hydrophobicity is created; and wherein when water mixes with the dye, the dye advances along the flow path zone; also urging said sample a distance along said substrate, the distance travelled along said substrate of the indicating dye providing a measure of concentration of fibrinogen in said blood sample under test.

31. A method of testing for the concentration of fibrinogen in a blood sample using a diagnostic device which enables measurement of fibrinogen concentration in the blood sample, the device comprising; a wettable testing substrate and a housing including viewing indicators which allow determination of a status of a test; the substrate having a first end and second end and intermediate therebetween a flow receiving zone, a flow path zone and a reaction zone; the reaction zone pre charged with reagents;
the method comprising the steps of:
a) pre charging said porous substrate with Thrombin chromogenic substrate and a dye/buffer solution, to provide a reagent reaction mixture in a reaction zone;
b) adding a blood or plasma sample near or in said receiving zone or near or in said reaction zone so that is engages with the reagent;
c) allowing the plasma to react with the thrombin to create a zone of hydrophobicity and induce clotting;
d) using the porosity in the substrate to transport the dye/buffer solution along the substrate and through the reaction zone,
e) observing a distance along the porous substrate that the dye/buffer solution passes;
f) determining fibrinogen concentration in said sample with reference to said distance that the dye/buffer solution travels along the flow path zone.

32. The method according to claim 31 comprising the further step of: allowing blood or plasma sample to form a clot and measuring an extent of clotting after initiating clotting of the sample, by measuring mass or volume or height of the clot.

33. The method according to claim 32 comprising the further step of; removing clotted blood or plasma from the non-porous substrate's surface by physical factors;

34. A testing method for determining the concentration of fibrinogen in a test sample using a porous substrate; the substrate having a first end and second end and intermediate therebetween a flow receiving zone, a flow path zone and a reaction zone; the method comprising the steps of:
g) pre charging said porous substrate with Thrombin chromogenic substrate to provide a reaction zone and an indicating dye;
h) adding a plasma sample to said reaction zone;
i) allowing the plasma to react with the thrombin to clot and create a zone of hydrophobicity;
j) washing the dye/buffer solution in the reaction zone,
k) observing a distance that the dye/buffer solution travels along the flow path zone and any colour change;
l) determining fibrinogen concentration in said sample with reference to said distance and/or said colour change.

35. The method according to claim 34 comprising the further step of quantifying the zone of hydrophobicity/hydrophobicity by measuring the distance travelled by at least one chromogenic marker through or away from the zone of hydrophobicity in a lateral flow.

36. The method of testing according to claim 35 comprising the additional step of quantifying the zone of hydrophobicity's surface hydrophobicity by measuring the shape, height and/or contact angle of any deposited liquid droplets on top of the non-porous substrate's surface.

37. The method according to claim 36 wherein the hydrophobicity is induced by the polymerization of the fibrinogen in the blood sample to fibrin upon enzymatic reaction with thrombin and/or FXIIIa deposited onto a receiving surface of the reaction zone.

38. The method according to claim 37 wherein diagnosis relies on a significant change of hydrophobicity induced by polymerization of the fibrinogen in the blood sample to fibrin upon enzymatic reaction with thrombin and/or FXIIIA deposited in the receiving zone.

39. The method according to claim 39 wherein said diagnosis of fibrinogen concentration can be made using the following combined three mechanisms;
1) a change in dye adhesiveness induced by polymerization of the fibrinogen in the blood sample to fibrin upon enzymatic reaction with thrombin and/or FXIIIa deposited onto the receiving zone;
2) adhesion of the dye deposited in the receiving zone to the fibrin directly (or indirectly via the assistance of dye binders); and
3) capillarity used to distribute the blood sample into the receiving zone treated with thrombin, and transport wash solution through the receiving surface to remove dye out of the porous material.

40. The method according to claim 39 wherein a colour intensity after washing is used to measure and visualize the quantity of fibrin-adhered dye remaining in the zone; thereby enabling a determination of fibrinogen concentration in the blood sample.

41. A method according to claim 40 wherein said Thrombin is Lyophillised.

42. A method according to claim 41 wherein the dye includes a buffer.
